(19) United States
(12) Patent Application Publication
(10) Pub. No.: US 2007/0019782 A1
(43) Pub. Date: Jan. 25, 2007

(54) Fan-beam coherent-scatter computed tomography

(76) Inventors: Udo Van Stevendaal, Ahrensburg (DE); Jens-Peter Schlomka, Hamburg (DE)

Correspondence Address: PHILIPS INTELLECTUAL PROPERTY & STANDARDS, P.O. BOX 3001, BRIARCLIFF MANOR, NY, 10510, US

(21) Appl. No.: 10/575,586

(22) PCT Filed: Oct. 5, 2004
(86) PCT No.: PCT/IB04/51967

§ 371 Date: Apr. 11, 2006

(30) Foreign Application Priority Data
Oct. 14, 2003 (EP) 031103789.8

(51) Int. Cl. A61B 6/00; G01N 23/00; G21K 1/12; H05G 1/60

(52) U.S. Cl. 378/6

(57) ABSTRACT

A reconstruction of data from CSCT data usually requires a long reconstruction time. According to the present invention, a filtered back-projection of CSCT data, wherein the CSCT data comprises a spectrum acquired by means of an energy resolving detector element is provided, wherein the filtered back-projection is performed along curved lines represented by the wave-vector transfer in the reconstruction volume. Advantageously, a reconstruction time may be reduced, while allowing for a good image quality.

The present invention relates to the field of coherent-scatter computed tomography (CSCT), where a fan-beam is applied to an object of interest. In particular, the present invention relates to a data processing device for performing a reconstruction of CSCT data, to a CSCT apparatus for examination of an object of interest, to a method of performing a reconstruction CSCT data and to a computer program for a data processor for performing a reconstruction of CSCT data.

U.S. Pat. No. 4,751,722 describes a device based on the principle of registration of an angled distribution of coherent scattered radiation within angles of 1° to 12° as related to the direction of the beam. As set forth in the U.S. Pat. No. 4,751,722, the main fraction of elastic scattered radiation is concentrated within angles of less than 12°, and the scattered radiation has a characteristic angle dependency with well marked maxima, the positions of which are determined by the irradiated substance itself. As the distribution of the intensity of the coherently scattered radiation in small angles depends on molecular structure of the substance, different substances having equal absorption capacity (which cannot be differentiated with conventional transillumination or CT) can be distinguished according to the distribution of the intensity of the angled scattering of coherent radiation typical for each substance.

Due to the improved capabilities of such systems to distinguish different object materials, such systems find more and more application in medical or industrial fields.

The dominant component of low-angle scatter is coherent scatter. Because coherent scatter exhibits interference effects which depend on the atomic arrangement of the scattering sample, coherent-scatter computed tomography (CSCT) is in principle a sensitive technique for imaging spatial variations in the molecular structure of tissues across a 2D object section.

Harding et al. “Energy-dispersive x-ray diffraction tomography” Phys. Med. Biol., 1990, Vol. 35, No. 1, 33-41 describes an energy dispersive x-ray diffraction tomograph (EXDT) which is a tomographic imaging technique based on an energy analysis at fixed angle, of coherent x-ray scatter excited in an object by polychromatic radiation. According to this method, a radiation beam is created by the use of suitable aperture systems, which has the form of a pencil and thus is also referred to as a pencil beam. Opposite to the pencil beam source, one detector element suitable for an energy analysis is arranged for detecting the pencil beam altered by the object of interest.

A coherent scatter set-up applying a fan-beam primary beam and a 2D detector in combination with CT was described in U.S. Pat. No. 6,470,067 B1. The shortcoming of the angle-dispersive set-up in combination with a polychromatic source are blurred scatter functions, which is described in e.g. Schneider et al. “Coherent Scatter Computed Tomography Applying a Fan-Beam Geometry” Proc. SPIE, 2001, Vol. 4320 754-763.

To become a competitive modality in the fields of medical imaging or non-destructive testing, the implemented reconstruction algorithm should assure both good image quality and short reconstruction times.

So far, the projection data acquired with fan-beam CSCT is reconstructed with the help for example, algebraic reconstruction techniques (ART), since ART has been shown to be highly versatile, for example, by J. A. Grant et al. “A reconstruction strategy suited to x-ray diffraction tomography” J. Opt. Soc. Am A12, 291-300 (1995). However, due to the computational complexity of such iterative reconstruction, such methods require a relatively long reconstruction time.

It is an object of the present invention to provide for a fast reconstruction of tomography data.

According to an exemplary embodiment of the present invention as set forth in claim 1, the above object may be solved with a data processing device for performing a reconstruction of CSCT data, wherein the CSCT data comprises a spectrum acquired by means of an energy resolving detector element. The data processing device comprises a memory for storing the CSCT data and a data processor for performing the filtered back-projection. The data processor is adapted to determine a wave-vector transfer by using the spectrum and to determine a reconstruction volume. Furthermore, according to an aspect of this exemplary embodiment, geometry information may be used to determine the reconstruction volume. A dimension of the reconstruction volume is determined by the wave-vector transfer. Hence, for example, one dimension of the reconstruction volume may be determined by the wave-vector transfer. The wave-vector transfer represents curved lines in the reconstruction volume. The data processor is furthermore adapted to perform a filtered back-projection along the curved lines in the reconstruction volume.

In the data processing device according to this exemplary embodiment of the present invention, a filtered back-projection is performed for CSCT data, comprising a spectrum acquired by means of an energy resolving detector element. Thus, a very precise and fast reconstruction of the CSCT data may be performed. In case the reconstructed data is represented in the form of images, the data processing device according to this exemplary embodiment of the present invention may allow for an improved image quality while keeping the reconstruction time relatively short. Furthermore, a precise reconstruction may be provided.

According to another exemplary embodiment of the present invention as set forth in claim 2, the spectrum data is acquired during a circular acquisition, where a source of radiation is rotated around an object of interest. Thus, for example, a CSCT scanner may be used for acquiring the data.

According to another exemplary embodiment of the present invention as set forth in claim 3, the two further dimensions (apart from the first dimension, which is determined by the wave-vector transfer) are determined by two linear independent vectors of the rotation plane of the source of radiation. In other words, the two further dimensions of the reconstruction volume are, for example, determined by coordinates relating to positions of the radiation source.

According to another exemplary embodiment of the present invention as set forth in claim 4, a preprocessing is performed to compensate for an attenuation contribution. This may allow for improved reconstruction.

According to another exemplary embodiment of the present invention as set forth in claim 5, a CSCT apparatus is provided for examination of an object of interest. The CSCT apparatus according to this exemplary embodiment of the present invention comprises a detector unit with an x-ray source and a scatter radiation detector. The detector unit is rotatable around a rotational axis extending through an examination area for receiving the object of interest The x-ray source generates a fan-shaped x-ray beam adapted to penetrate the object of interest in the examination area in a slice plane. The scatter radiation detector is arranged at the detector unit opposite the x-ray source with an offset with respect to the slice plane in a direction parallel to the rotational axis. The scatter radiation detector includes a first detector line with a plurality of first detector elements arranged in a line. The plurality of first detector elements are either energy resolving detector elements or integrating (non-energy resolving) detector elements. Furthermore, there is provided a data processor for performing a filtered back-projection on first read-outs of the scatter radiation detector, wherein the data processor is adapted to determine a wave-vector transfer by using the first read-outs. Furthermore, the data processor is adapted to determine a reconstruction volume. A dimension of the reconstruction volume is determined by the wave-vector transfer. Furthermore, the wave-vector transfer represents curved lines in the reconstruction volume. Furthermore, the data processor is adapted to perform a filtered back-projection along the curved lines in the reconstruction volume.

Advantageously, according to this exemplary embodiment of the present invention, a CSCT apparatus may be provided, allowing for an improved image quality of the reconstructed images, while allowing for a reduced reconstruction time.

Further exemplary embodiments of the CSCT apparatus are provided in claims 6 and 7.

According to another exemplary embodiment of the present invention as set forth in claim 8, a method of performing a reconstruction of CSCT data is provided, wherein the CSCT data comprises a spectrum acquired by means of an energy resolving detector element. According to this method, a wave-vector transfer is determined by using the spectrum. Then, a reconstruction volume is determined. A dimension of the reconstruction volume is determined by the wave-vector transfer. The wave-vector transfer represents curved lines in the reconstruction volume. According to an aspect of this exemplary embodiment of the present invention, the filtered back-projection is performed along the curved line in the reconstruction volume.

Advantageously, this method may allow for a reduction of the reconstruction time. Furthermore, this method may allow for an exact reconstruction of, for example, CSCT data.

Further exemplary embodiments of the method according to the present invention are provided in claims 9 to 12.

According to another exemplary embodiment of the present invention as set forth in claim 13, a computer program for a data processor for performing a filtered back-projection of CSCT data is provided. The computer program according to the present invention is preferably loaded into a working memory of the data processor. The data processor is thus equipped to carry out the method of the invention. The computer program may be stored on a computer readable medium, such as a CD-Rom. The computer program may also be presented over a network such as the Worldwide Web, and can be down-loaded into the working memory of a data processor from such a network.

It may be seen as the gist of an exemplary embodiment of the present invention that a filtered back-projection is performed along curved lines, which deals with data acquired by energy-resolving detector lines. The dependence of the scattered photons on the wave-vector transfer is calculated from the energy dependence. The data is interpreted as integrals along the curved lines in the reconstruction space, such as the x-y-q space. According to an aspect of the present invention, the result is a 3-D data set, yielding the spatially resolved scatter function of one illuminated object slice. The present invention may, for example, be applied in medical imaging or material analysis, for example, in baggage inspection. Advantageously, a very fast image reconstruction may be performed, for example, in CSCT, where just one row of energy resolving detector elements is used.

These and other aspects of the present invention will become apparent from and elucidated with reference to the embodiments described hereinafter.

Exemplary embodiments of the present invention will be described in the following, with reference to the following drawings:

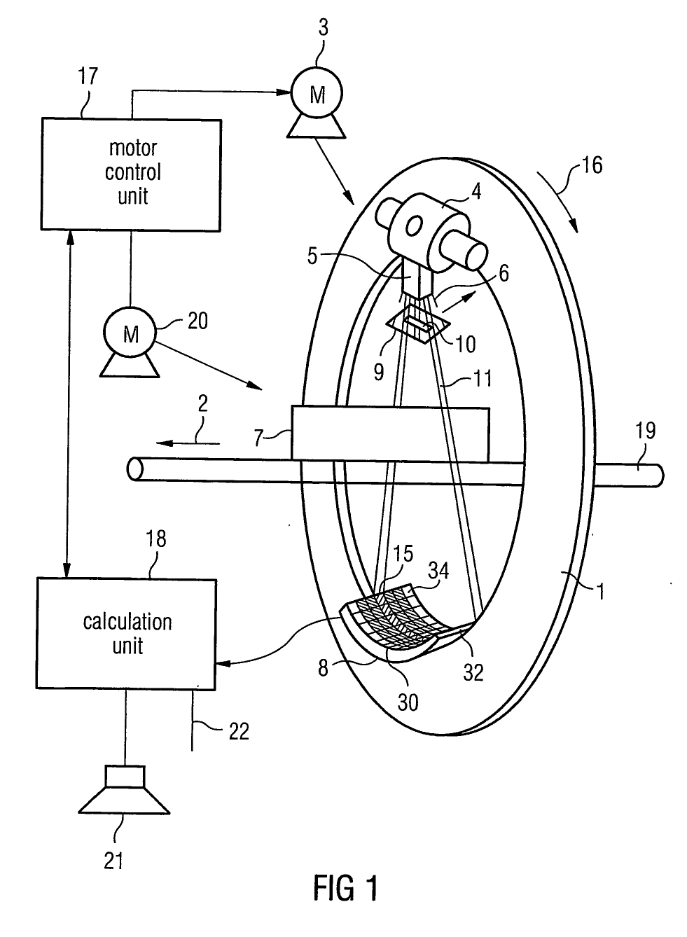

FIG. 1 shows a schematic representation of an exemplary embodiment of a CSCT scanner according to the present invention.

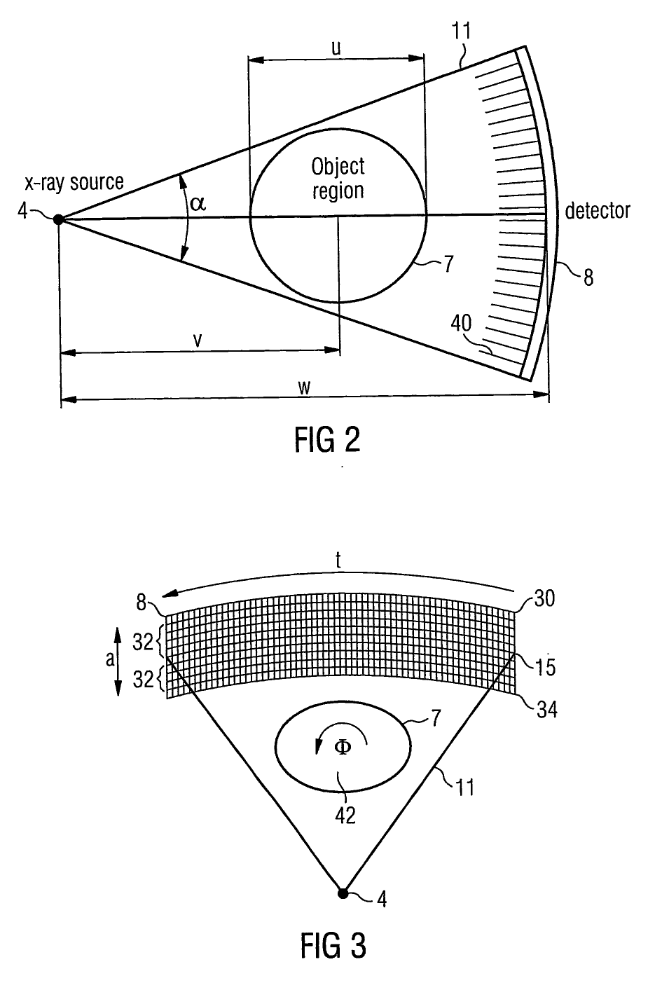

FIG. 2 shows a schematic representation of the geometry of the CSCT scanner of FIG. 1 for the measurement of coherent scatter radiation.

FIG. 3 shows another schematic representation of the geometry of the CSCT scanner of FIG. 1.

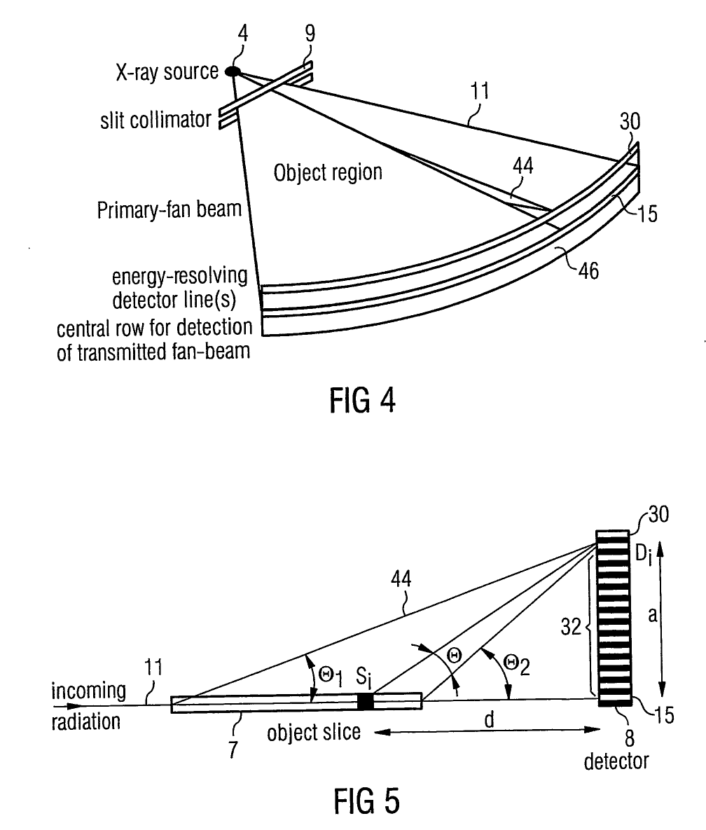

FIG. 4 shows another schematic representation of the measurement geometry of the CSCT scanner of FIG. 1 for further explaining the present invention.

FIG. 5 shows a schematic representation of a side view of the geometry of the CSCT scanner of FIG. 1.

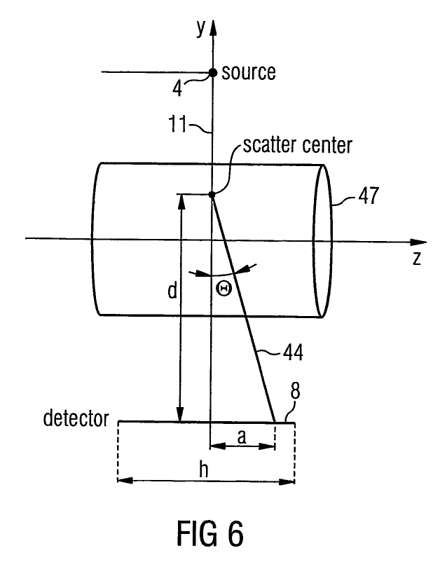

FIG. 6 shows a simplified schematic representation of a possible scanner geometry for performing a filtered back-projection of CSCT data according to the present invention.

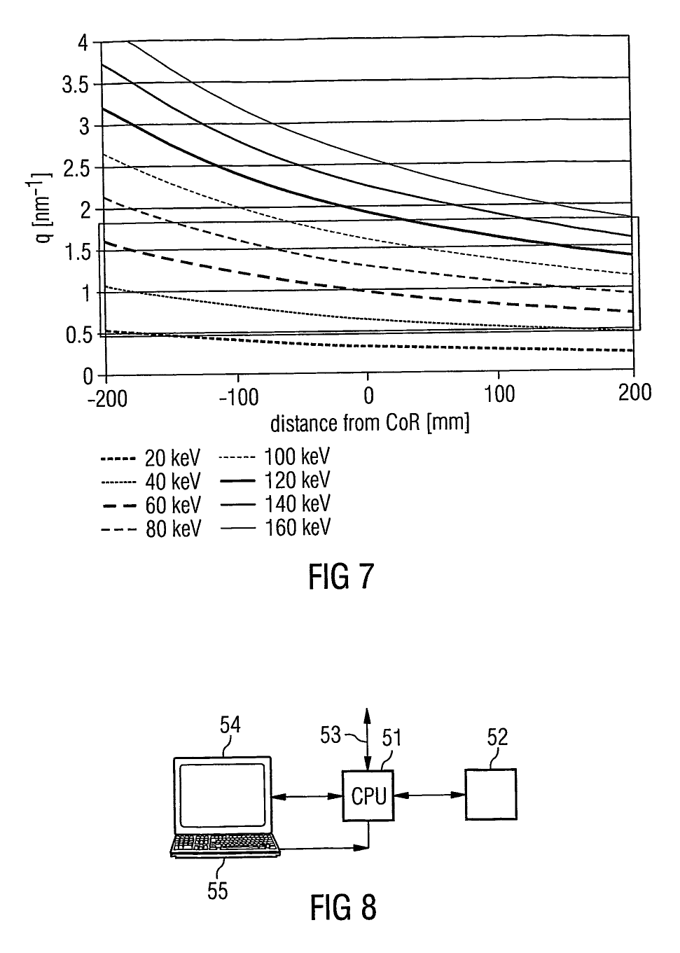

FIG. 7 shows a relation between a position in the object of interest and the wave-vector transfer for various energies for further explaining the present invention.

FIG. 8 shows a simplified schematic representation of an exemplary embodiment of a data processing device according to the present invention.

FIG. 1 shows an exemplary embodiment of CSCT scanner according to the present invention. With reference to this exemplary embodiment, the present invention will be described for the application in baggage inspection to detect hazardous materials such as explosives in items of baggage. However, it has to be noted that the present invention is not limited to applications in the field of baggage inspection, but can also be used in other industrial or medical applications, such as for example in bone imaging or a discrimination of tissue types in medical applications.

The scanner depicted in FIG. 1 is a fan-beam CSCT scanner, which allows in combination with an energy-resolving detector and with tomographic reconstruction a good spectral resolution, even with a polychromatic primary fan-beam. The CSCT scanner depicted in FIG. 1 comprises a gantry 1, which is rotatable around a rotational axis 2. The gantry 1 is driven by means of a motor 3. Reference character 4 designates a source of radiation, such as an x-ray source, which, according to and aspect of the present invention, emits a polychromatic radiation.

Reference character 5 designates a first aperture system, which forms the radiation beam emitted from the radiation source 4 to a cone shaped radiation beam 6. Furthermore, there is provided another aperture system 9 consisting of a diaphragm or a slit collimator. The aperture system 9 has the form of a slit 10, such that the radiation emitted from the source of radiation 4 is formed into a fan-beam 11. According to a variant of this exemplary embodiment of the present invention, the first aperture system 5 may also be omitted and only the second aperture 9 may be provided.

The fan-beam 11 is directed such that it penetrates the item of baggage 7, arranged in the center of the gantry 1, i.e. in an examination region of the CSCT scanner and impinges onto the detector 8. As may be taken from FIG. 1, the detector 8 is arranged on the gantry 1 opposite to the radiation source 4, such that the slice plane of the fan-beam 11 intersects a row or line 15 of the detector 8. The detector 8 depicted in FIG. 1 has seven detector lines, each comprising a plurality of detector elements. As mentioned above, the detector 8 is arranged such that the primary radiation detector 15, i.e. the middle line of the detector 8 is in the slice plane of the fan-beam 11.

As can be taken from FIG. 1, the detector 8 comprises two types of radiation detector lines: a first type of detector lines 30 and 34, which are indicated without hatching in FIG. 1, which are detector lines consisting of energy resolving detector cells. According to an aspect of the present invention, these first detector elements (lines 30 and 34) are energy-resolving detector elements. Preferably, the energy resolving detector elements are direct-converting semiconductor detectors. Direct-converting semiconductor detectors directly convert the radiation into electrical charges—without scintillation. Preferably, these direct-converting semiconductor detectors have an energy resolution better than 10% FWHM, i.e. ΔE/E<0.1, with ΔE being the full-width at half maximum (FWHM) of the energy resolution of the detector.

Such detector cells of lines 30 and 34 may be cadmiumtelluride or CdZnTe (CZT) based detector cells, which are both outside of the slice plane of the fan-beam 11. In other words, both energy resolving lines 30 and 34 are arranged at the gantry 1 opposite to the x-ray source 4 with an offset from the slice plane in a direction parallel to the rotational axis 2. The detector line 30 is arranged with a positive offset with respect to the direction of the rotational axis 2 depicted in FIG. 1, whereas the line 34 is arranged with a negative offset from the slice plane with respect to the direction of the rotational axis 2 depicted in FIG. 1.

The detector lines 30 and 34 are arranged at the gantry 1 such that they are parallel to the slice plane and out of the slice plane with an offset in a positive or negative direction of the rotational axis 2 of the gantry 1, such that they receive or measure a scatter radiation scattered from the item of baggage 7 in the examination area of the CSCT scanner. Thus, in the following, lines 30 and 34 will also be referred to as scatter radiation detector. It has to be noted that instead of the provision of two energy resolving lines 30 and 34, it may also be efficient to provide only one line which includes energy resolving detector elements, such as, for example, only the line 30. Furthermore, instead of providing only two energy resolving lines 30 and 34, it is also possible to provide three, four or an even greater number of energy resolving lines. Thus, if, in the following the term “scatter radiation detector” is used, it includes any detector with at least one line of energy resolving detector cells, which is arranged out of the fan plane of the fan-beam 11, such that it receives photons scattered from the item of baggage 7.

The second type of detector lines provided on the detector 8, which are indicated by a hatching, are scintillator cells. In particular, line 15 is arranged such that it is in the slice plane of the fan-beam 11 and measures the attenuation of the radiation emitted by the source of radiation 4, caused by the item of baggage 7 in the examination area. As depicted in FIG. 1, right and left of the line 15, there may be provided further detector lines including scintillator detector cells.

As already indicated with respect to the energy resolving lines 30 and 34, where the provision of only one energy resolving line 30 or 34 is sufficient, the provision of only the line 15 measuring the attenuation caused by the item of baggage 7 of the primary beam of the fan-beam 11 in the slice plane is sufficient. However, as in the case of the energy resolving lines 30 and 34, a provision of a plurality of detector lines 32, each comprising a plurality of scintillator cells, may further increase the measurement speed of the CSCT scanner. In the following, the term “primary radiation detector” will be used to refer to a detector, including at least one line of scintillator or similar detector cells for measuring an attenuation of the primary radiation of the fan-beam 11.

As may be taken from FIG. 1, the detector cells of the detector 8 are arranged in lines and columns, wherein the columns are parallel to the rotational axis 2, whereas the lines are arranged in planes perpendicular to the rotational axis 2 and parallel to the slice plane of the fan-beam 11.

The apertures of the aperture systems 5 and 9 are adapted to the dimensions of the detector 8 such that the scanned area of the item of baggage 7 is within the fan-beam 11 and that the detector 8 covers the complete scanning area. Advantageously, this allows to avoid unnecessary excess radiation applied to the item of baggage 7. During a scan of the item of baggage 7, the radiation source 4, the aperture systems 5 and 9 and the detector 8 are rotated along the gantry 1 in the direction indicated with arrow 16. For rotation of the gantry 1 with the source of radiation 4, the aperture systems 5 and 9 and the detector 15, the motor 3 is connected to a motor control unit 17, which is connected to a calculation unit 18.

In FIG. 1, the item of baggage 7 is disposed on a conveyor belt 19. During the scan of the item of baggage 7, while the gantry 1 rotates around the item of baggage 7, the conveyor belt 19 displaces the item of baggage 7 along a direction parallel to the rotational axis 2 of the gantry 1. By this, the item of baggage 7 is scanned along a helical scan path. The conveyor belt 19 can also be stopped during the scans to thereby measure single slices.

The detector 8 is connected to a calculation unit 18. The calculation unit 18 receives the detection results, i.e. the readouts from the detector elements of the detector 8 and determines a scanning result on the basis of the scanning results from the detector 8, i.e. from the energy resolving lines 30 and 34 and the lines 15 and 32 for measuring the attenuation of the primary radiation of the fan-beam 11. In addition to that, the calculation unit 18 communicates with the motor control unit 17 in order to coordinate the movement of the gantry 1 with the motors 3 and 20 or with the conveyor belt 19.

The calculation unit 18 is adapted for reconstructing an image from readouts of the primary radiation detector, i.e. detector lines 15 and 32 and the scatter radiation detector, i.e. lines 30 and 34. The image generated by the calculation unit 18 may be output to a display (not shown in FIG. 1) via an interface 22.

The calculation unit, which may be realized by a data processor, may be adapted to perform a filtered back-projection on the read-outs from the detector element of the detector 8, i.e. from the read-outs from the energy resolving lines 30 and 34 and the lines 15 and 32 for measuring the attenuation of the primary radiation of the fan-beam 11. The back-projection performed in the calculation unit 18, which forms part of the image reconstruction will be described in further detail with reference to FIG. 7.

Furthermore, the calculation unit 18 may be adapted for the detection of explosives in the item of baggage 7 on the basis of the readouts of the lines 30 and 34 and 15 and 32. This can be made automatically by reconstructing scatter functions from the readouts of these detector lines and comparing them to tables including characteristic measurement values of explosives determined during preceding measurements. In case the calculation unit 18 determines that the measurement values read out from the detector 8 match with characteristic measurement values of an explosive, the calculation unit 18 automatically outputs an alarm via a loudspeaker 21.

During the subsequent description of FIGS. 2 to 7, the same reference numbers as used in FIG. 1 will be used for the same or corresponding elements.

FIG. 2 shows a simplified schematic representation of a geometry of the CSCT scanning system depicted in FIG. 1. As may be taken from FIG. 2, the x-ray source 4 emits the fan-beam 11 such that it includes the item of baggage 7 in this case having a diameter of u and covers the entire detector 8. The diameter of the object region may, for example, be 100 cm. In this case, an angle α of the fan-beam 11 may be 80°. In such an arrangement, a distance v from the x-ray source 4 to the center of the object region is approximately 80 cm and the distance of the detector 8, i.e. of the individual detector cells from the x-ray source 4 is approximately w=150 cm.

As can be taken from FIG. 2, according to an aspect of the present invention, the detector cells or lines can be provided with collimators 40 to avoid that the cells or lines measure unwanted radiation having a different scatter angle. The collimators 40 have the form of blades or lamellae, which can be focused towards the source. The spacing of the lamellae can be chosen independently from the spacing of the detector elements.

Instead of a bent detector 8 as depicted in FIGS. 1 and 2, it is also possible to use a flat detector array.

FIG. 3 shows another schematic representation of a detector geometry as used in the CSCT scanner of FIG. 1. As already described with reference to FIG. 1, the detector 8 may comprise one, two or more energy resolving detector lines 30 and 34 and a plurality of lines 15 and 32 for measuring the attenuation of the primary fan-beam caused by the item of baggage 7. As may be taken from FIG. 3, preferably the detector 8 is arranged such that one line of the lines 15 and 32, preferably the middle line 15 of the detector 8, is within the slice plane of the fan-beam 11 and thereby measures the attenuation in the primary radiation. As indicated by arrow 42, the x-ray source 4 and the detector 8 are rotated together around the item of baggage to acquire projections from different angles.

As depicted in FIG. 3, the detector 8 comprises a plurality of columns t.

FIG. 4 shows another schematic representation of the geometry of the CSCT scanner depicted in FIG. 1 for further explaining the present invention. In FIG. 4, a detector 46 is depicted, comprising only one line 15 and only one line 30. The line 15 is arranged in the slice plane of the fan-beam 11 formed by the aperture system 9, which in this case is a slit collimator and generated by means of the source of radiation or x-ray source 4. The line 15 comprises, for example, scintillator cells or other suitable cells for measuring the attenuation of the primary beam of the fan-beam 11 and allows for an integral measurement of the attenuation of the primary fan-beam caused by the object of interest in the object region or examination region.

Line 30 depicted in FIG. 4 includes energy resolving cells. As may be taken from FIG. 4, the line 30 is arranged parallel to the slice plane of the fan-beam 11 but out of the plane. In other words, the line 30 is arranged in a plane parallel to the slice plane and parallel to the line 15.

Reference numeral 44 indicates a scatter radiation, i.e. a photon scattered by the object of interest, such as the item of baggage. As may be taken from FIG. 4, the scatter radiation leaves the slice plane and impinges onto a detector cell of the line 30.

FIG. 5 shows a side view of the detector geometry of the CSCT scanner of FIG. 1. FIG. 5 can also be contemplated as showing a side view of FIG. 4, where, however, instead only the provision of one line 30 and one line 15, in FIG. 5, there is provided a plurality of detector lines 32 between the line 30 and the line 15. The detector element Dᵢ of the line 30 is an energy resolving detector element. The detector element Dᵢ is arranged with a fixed distance a from the slice plane of the primary fan-beam. According to an aspect of the present invention, for each detector element Dᵢ of the column t and for each projection Φ (see FIG. 3) a spectrum I(E, t, Φ) is measured. Performing this measurement for a plurality of projections Φ along a circular or helical scan path, a three-dimensional dataset is acquired. Each object pixel is described by three coordinates (x, y, q). Thus, according to an aspect of the present invention, for reconstructing an image or for reconstructing further information from the three-dimensional dataset, a 3D→3D reconstruction method can be used such as the one described in DE 10252662.1, which is hereby incorporated by reference.

On the basis of the spatial coordinates (x, y), a distance d of each object voxel Sᵢ to the detector 8 is calculated by means of the calculation unit 18. Then, the calculation unit 18 calculates a scatter angle Θ for each object voxel Sᵢ and spaces of the following equation:
Θ=a tan (a/d) (Equation 1).

Then, on the basis of this calculation, the calculation unit 18 calculates the wave-vector transfer parameter q on the basis of the following equation:

$$q = \frac{E}{hc} sin ( Θ / 2 ) , \qquad ( Equation 2 )$$

wherein h is the Planck's constant, c is the speed of light and E the photon energy.

Then, on the basis of the wave-vector transfer parameter q calculated in accordance with the above formulas and on the basis of the readouts of the primary radiation detector (for attenuation correction) and the scattered radiation data, the calculation unit 18 may determine an image or may discriminate the material in the object slice.

FIG. 6 shows the geometry of the scanner of FIG. 1 to better illustrate the scattering process. As may be taken from FIG. 6, the scattering process takes place at a scatter center, such that the scatter radiation 44 is scattered out of the x-y plane of the fan-beam 11. The cylinder 47 symbolizes the object around which the source of radiation 4 rotates.

In the following, the filtered back-projection according to an exemplary embodiment of the present invention, which, as indicated above, may be performed in the calculation unit 18 or in the data processing device depicted in FIG. 8, is described in further detail.

For a dispersion with small angles, i.e. angles between 0° and approximately 5° sin (Θ/2) may be approximated by Θ/2. Due to this, the Equation 2 may be written as follows:

$$q \approx \frac{E}{hc} \frac{Θ}{2} . \qquad ( Equation 3 )$$

As may be taken, for example, from FIG. 6, the dispersion angle may be determined on the basis of the distance d from the scatter center to the detector and the distance a of the respective detector element or line to the scanning plane or fan-beam plane. Hence, Equation 3 may be written as follows:

$$tan Θ \approx Θ = \frac{a}{d} . \qquad ( Equation 4 )$$

A combination of Equations 3 and 4 gives

$$q = \frac{E}{hc} \frac{a}{2 d} . \qquad ( Equation 5 )$$

Equation 5 describes curved lines such as hyperbolas in the x-y-q space, which, in the following, is referred to as reconstruction volume. Then, according to an aspect of the present invention, the filtered back-projection is performed along these hyperbolas or other corresponding curved lines.

In other words, as described above, a wave-vector transfer q is determined by using the spectrum E. Then, a reconstruction volume is determined.

According to an aspect of the present invention, the reconstruction volume is determined by the coordinates x and y in the rotation plane of the radiation source or in the fan-beam plane. The dimensions x and y may be represented by vectors. Preferably, these vectors are linear and independent vectors.

The third dimension of the reconstruction volume is determined by the wave-vector transfer q itself, thus forming the x-y-q reconstruction volume. As shown by Equation 5, the wave-vector transfer represents curved lines such as hyperbolas in the reconstruction volume. Then, according to the present invention, the filtered back-projection is performed along the curved lines in the reconstruction volume.

According to an aspect of the present invention, the filtering may be performed as described, for example, by Kak et al. in “Principles of Computerized Tomographic Imaging” (IEEE, New York, 1988), which is hereby incorporated by reference.

Before the filtered back-projection described with Equations 1 to 5, i.e. before reconstruction, a preprocessing step of the scatter projection data may be performed in order to compensate for the attenuation contribution. In the following, the variables α and β denote the angular source position in relation to the x axis and the fan-angle within the fan-beam of x-rays. Furthermore, l₀ is the distance from the x-ray source to the scatter center.

The factor A(α,β,0,l₀) accounts for the attenuation of the incoming radiation along the path from the source to the point of interaction x₀. The factor B(α,β,a,l₀) is the analogous attenuation for the outgoing radiation. According to an aspect of the present invention, an assumption is made, namely that the attenuation along the path of the scattered radiation is independent of the scattering angle and equal to the attenuation of the residual primary beam B(α,β,a,l₀)=B(α,β,0,l₀).

This holds true for small scatter angles, i.e. scatter angles in the approximate range of 0° to 5°. Also, this holds true for ideal spatial resolution and not too strong variations of the attenuation along the z direction. For an attenuation correction, the transmitted intensities Iₜᵣₐₙₛ and the detector elements of the central plane (i.e. the primary radiation detector; detector line 15), in case of a simple transmission CT, are taken into account:
Iₜᵣₐₙₛ(α,β,0,l₀)=I₀(α,β,0)A(α,β,0,l₀)×B(α,β,0,l₀)E_{CT}(α,β,0)
with the intensity I₀ of the incoming radiation and a constant geometrical efficiency E_{CT}(α,β,0)=A/G². Here G and A denote the distance from the x-ray source to the focus-centered detector and the area of a single detector element, respectively.

This leads to the scatter projection data P_{D}(α,β,a) as input for the reconstruction algorithm according to U. van Stevendaal et al., “A reconstruction algorithm for coherent scatter computed tomography based on filtered back-projection” (Med. Phys. 30, 9, Sep. 2003),

$${P}_{D} ( α , β , a ) = \frac{{\int }_{0}^{G} {I}_{coh} ( α , β , a , {l}_{0} ) ⅆ {l}_{0}}{{\int }_{0}^{G} {I}_{trans} ( α , β , 0 ) ⅆ {l}_{0}} \\ = {\int }_{0}^{G} {\vert F ( α , β , a , {l}_{0} ) \vert}^{2} ξ ( α , β , a , {l}_{0} ) ⅆ {l}_{0}$$

with the overall efficiency ξ(α,β,a,l₀)=E_{eff}(α,β,a,l₀)/E_{CT}(α,β,0) or

$$ξ ( α , β , a , {l}_{0} ) = \frac{{G}^{2} ( G - {l}_{0} )}{{( {h}^{2} + {( G - {l}_{0} )}^{2} )}^{3 / 2}} ,$$

where E_{eff}(≢,β,a,l₀) is the geometrical efficiency factor for an off-plane detector element.

Advantageously, the projection data of coherently scattered x-rays may not be corrected concerning the attenuation contribution. Furthermore, an overall efficiency is introduced in order to weight the projection data more accurately.

FIG. 7 shows a relation between a position in the object (here a distance from the center of rotation “CoR”) and the wave-vector transfer for various energies. The distance a is 20 mm and the distance between CoR and the center of the detector is approximately 500 mm. As may be taken from FIG. 8, in case a radiation is detected in the range from 20 to 160 keV, then, for an object having a diameter of 400 mm, a complete dataset may be obtained for a wave-vector transfer of 0.5 to 1.8 nm⁻¹. In particular in the case of material discrimination, this range is advantageous, since most of the structures used for material identifications are in this range.

FIG. 8 shows an exemplary embodiment of a data processing device for performing a filtered back-projection of CSCT data, for example, in the same manner as described with reference to FIG. 6 and also the preprocessing described above. As may be taken from FIG. 8, a central processing unit (CPU) or image processor 1 is connected to a memory 2 for storing the CSCT data, which may be acquired by a CSCT scanner such as the one depicted in FIG. 1. The image or data processor may be connected to a plurality of input/output-, network-, or diagnosis devices such as an MR device. The data processor 1 is furthermore connected to a display 4 (for example, to a computer monitor) for displaying information or images computed or adapted in the data processor 1. An operator may interact with the data processor 1, via a keyboard 5 and/or other output devices, which are not depicted in FIG. 1.

As indicated above, the data processor is adapted for performing the filtered back-projection, involving a determination of the wave-vector by using the spectrum determined by energy resolving detector elements of a CT scanner. Then, a reconstruction volume is determined, where one dimension of the reconstruction volume is determined by the wave-vector transfer and the remaining two dimensions may be determined by the position coordinates in the fan-beam plane or the rotation plane of a source of radiation of the CT scanner. As indicated in Equation 5 above, the wave-vector transfer may be interpreted as curved lines, such as hyperbolas, in the reconstruction volume. Then, the filtered back-projection is performed along the curved lines in the reconstruction volume.

Advantageously, the present invention allows for a very fast reconstruction. In case images are reconstructed from the CSCT data, the images may have an improved quality. As mentioned above, it may be sufficient to provide only one row of energy resolving detector elements. However, with more than one row of energy detector elements, a broader spectrum of q values may be acquired and the scanning time may be reduced.

1. Data processing device for performing a reconstruction of CSCT data, wherein the CSCT data comprises a spectrum acquired by means of an energy resolving detector element, the data procession device comprising: a memory for storing the CSCT data; and a data processor for performing a filtered back-projection, wherein the data processor is adapted to perform the following operation: determining a wave-vector transfer by using the spectrum; determining a reconstruction volume; wherein a dimension of the reconstruction volume is determined by the wave-vector transfer, wherein the wave vector transfer represents curved lines in the reconstruction volume; and performing a filtered back-projection along the curved lines in the reconstruction volume.

2. The data processing device of claim 1, wherein the spectrum is acquired during a circular acquisition where a source of radiation is rotated around an object of interest in a rotation plane.

3. The data processing device of claim 2, wherein the reconstruction volume is furthermore determined by two linear independent vectors of the rotation plane.

4. The data processing device of claim 1, wherein the energy resolving detector is arranged such that it measures a scatter radiation scattered by an object of interest; wherein the CSCT data further comprises information with respect to a primary radiation attenuated by the object of interest; and wherein a preprocessing is performed to correct for an attenuation contribution.

5. A CSCT apparatus for examination of an object of interest, the CSCT apparatus comprising: a detector unit with an x-ray source and a scatter radiation detector; wherein the detector unit is rotatable around a rotational axis extending through an examination area for receiving the object of interest; wherein the x-ray source generates a fan-shaped x-ray beam adapted to penetrate the object of interest in the examination area in a slice plane; wherein the scatter radiation detector is arranged at the detector unit opposite to the x-ray source with an offset with respect to the slice plane in a direction parallel to the rotational axis; wherein the scatter radiation detector includes a first detector line with a plurality of first detector elements arranged in a line; wherein the plurality of first detector elements are energy-resolving detector elements; a data processor for performing a filtered back-projection on first readouts of the scatter radiation detector, wherein the data processor is adapted to perform the following operation: determining a wave-vector transfer by using the first readouts; determining a reconstruction volume; wherein a dimension of the reconstruction volume is determined by the wave-vector transfer; wherein the wave-vector transfer represents curved lines in the reconstruction volume; and performing a filtered back-projection along the curved lines in the reconstruction volume.

6. The CSCT apparatus according to claim 5, wherein the scatter radiation detector is arranged at the detector unit opposite to the x-ray source parallel to the slice plane and out of the slice plane with such an offset along the rotational axis such that the scatter radiation detector is arranged for receiving a scatter radiation scattered from the object of interest, and wherein the CSCT apparatus further comprises: a primary radiation detector; wherein the primary radiation detector is arranged at the detector unit opposite to the x-ray source in the slice plane for receiving a primary radiation attenuated by the object of interest; and wherein the data processor performs a preprocessing to correct for an attenuation contribution by using second readouts of the primary radiation detector.

7. The CSCT apparatus according to claim 5, wherein the reconstruction volume is furthermore determined by two linear independent vectors of the rotation plane and a wave-vector transfer dimension.

8. Method of performing a reconstruction of CSCT data, wherein the CSCT data comprises a spectrum acquired by means of an energy resolving detector element, the method comprising the steps of: determining a wave-vector transfer by using the spectrum; determining a reconstruction volume; wherein a dimension of the reconstruction volume is determined by the wave-vector transfer, wherein the wave-vector transfer represents curved lines in the reconstruction volume; and performing a filtered back-projection along the curved lines in the reconstruction volume.

9. The method of claim 8, wherein the spectrum is acquired during a circular acquisition where a source of radiation is rotated around an object of interest in a rotation plane.

10. The method of claim 9, wherein the reconstruction volume is furthermore determined by two linear independent vectors of the rotation plane.

11. The method of claim 8, wherein the energy resolving detector is arranged such that it measures a scatter radiation scattered by an object of interest; wherein the CSCT data further comprises information with respect to a primary radiation attenuated by the object of interest; and wherein a preprocessing is performed to correct for an attenuation contribution.

12. The method of claim 8, further comprising the steps of: energizing an x-ray source such that it generates a fan-shaped x-ray beam which penetrates the object of interest in an examination area in a slice plane; performing an integral energy measurement of a scatter radiation by means of a scatter radiation detector with a first detector line with a plurality of first energy-resolving detector elements arranged in a line; reading-out the energy measurement from the scatter radiation detector; rotating the x-ray source and the scatter radiation detector around a rotational axis extending through an examination area containing the object of interest.

13. Computer program for a data processor for performing a reconstruction of CSCT data, wherein the CSCT data comprises a spectrum acquired by means of an energy resolving detector element, wherein the computer program causes the data processor to perform the following operation: determining a wave-vector transfer by using the spectrum; determining a reconstruction volume; wherein a dimension of the reconstruction volume is determined by the wave-vector transfer, wherein the wave-vector transfer represents curved lines in the reconstruction volume; and performing a filtered back-projection along the curved lines in the reconstruction volume.
